(19) United States
(12) Patent Application Publication
(10) Pub. No.: US 2004/0241808 A1
(43) Pub. Date: Dec. 2, 2004

(54) Methods for ex vivo hybridoma-free production of polyclonal and monoclonal antibodies and generation of immortalized cell populations

(76) Inventors: Renata Pasqualini, Houston, TX (US); Wadih Arap, Houston, TX (US)

Correspondence Address: FULBRIGHT & JAWORSKI L.L.P., 600 CONGRESS AVE., SUITE 2400, AUSTIN, TX, 78701, US

(21) Appl. No.: 10/824,627

(22) Filed: Apr. 14, 2004

Related U.S. Application Data
(60) Provisional application No. 60/462,631, filed on Apr. 14, 2003.
(60) Provisional application No. 60/524,701, filed on Nov. 24, 2003.

(51) Int. Cl. C12Q 1/68; C12P 21/04; C12N 5/06

(52) U.S. Cl. 435/70.21; 435/326; 530/387.1

(57) ABSTRACT

The invention includes methods and compositions for the generation of antibody-producing cells without the need for hybridoma formation. In one embodiment, the method includes the generation of monoclonal antibody producing cells by harvesting cells from a transgenic mouse with an immortalized cell population. In other embodiments, the method includes the use of a transgenic mouse with an immortalized cell population crossed with a transgenic mouse capable of producing humanized antibodies to produce a human and immortalized monoclonal antibody producing cell line.

This application claims priority to U.S. Provisional Patent Application Nos. 60/462,631 filed on Apr. 14, 2003 and 60/524,701 filed on Nov. 24, 2003, which are hereby incorporated by reference in their entirety.

### BACKGROUND OF THE INVENTION

1. Field of the Invention

The present invention concerns the fields of molecular biology, cellular biology, and immunology. More specifically, the present invention relates to methods and uses for synthesis of antibodies without the use of hybridomas.

2. Background

Monoclonal antibodies are proteins with high specificity and sensitivity in their reactions with specific sites on target molecules. Monoclonal antibodies over the years have become reagents of central importance in modern biological research and medicine, such as the analysis and treatment of human disease. However, more than a quarter century after their introduction, monoclonal antibodies are still produced only by somatic cell clones of splenocytes fused to multiple myeloma-derived cells (hybridomas) (Kohler and Milstein, 1975). These “hybridomas” are capable of producing monoclonal antibodies for years, but production involves a labor-intensive multi-step process that is limited by the constant risk of contamination, frequent requirement of feeder cells, as well as possible genetic instability (Harlow and Lane, 1988). The process of hybridoma production is rarely completed in two months and often takes well over one year.

The traditional approach for generating monoclonal antibodies suffers from at least two limitations: (i) the lack of stability of hybridoma cell lines due largely to genetic instability and (ii) the limited time for the selection and screening of clones since the hybridomas have a duration of only 2-3 weeks in culture and must be screened within this time for specificity of binding. Despite recent technological developments such as phage display technology for in vitro generation of monoclonal antibodies (Winter et al., 1994; Barbas et al., 2000), chimerization or humanization strategies (Winter and Milstein, 1991), and human myeloma cell lines suitable for hybridoma formation (Karpas et al., 2001), additional strategies and methods for the generation of an immortal monoclonal antibody-producing cells are still needed and would represent a major advance in the field.

### SUMMARY OF THE INVENTION

Embodiments of the invention include methods for generating an antibody-producing cell that produces an antibody to a desired antigen without having to fuse the antibody producing cell to an immortalize cell, e.g., hybridoma production. Aspects of the invention include the steps of contacting antibody-producing cells with the desired antigen in a manner effective to induce the cells to produce antibodies against the antigen, wherein the antibody-producing cells are capable of being immortalized without forming hybridomas; and immortalizing the antibody-producing cell. The antibody-producing cell typically comprise a transforming oncogene that is conditionally functional or conditionally expressed, and the immortalization of the antibody-producing cell is effected by induction of the expression or function of the transforming oncogene (induction of the transforming oncogene). In certain embodiments, the conditionally functional transforming oncogene is a temperature sensitive SV40 Large Tumor antigen (tsSV40Tag), preferably the tsSV40Tag is an A58S-SV40Tag. In certain aspects of the invention the transforming oncogene is induced by culturing the antibody producing cells in a temperature range from 25° C. to 35° C., preferably from 30° C. to 35° C., and more preferably at about 33° C. Typically, the antibody-producing cells are cultured in hybridoma culture medium.

In still further embodiments, the methods further comprise assessing the antibody producing capabilities of the antibody-producing cells. Assessment of the antibody-producing cells may comprise assaying antibody binding to said desired antigen, as well as similar and dissimilar antigens. Single cells are typically selected and cultured to produce a monoclonal cell population that produce monoclonal antibodies. Single cells may be selected by dilution cloning. In other aspects of the invention, multiple cells are selected and cultured to produce a polyclonal cell population that produce polyclonal antibodies. In certain embodiments, the antibody-producing cell comprise spleen cells (splenocytes). Antigens may be peptides; proteins; glycoproteins; lipoproteins; carbohydrates; viruses; bacteria; pathogenic microorganisms; tissue; whole cells; biopsy tissue; patient-derived cells; tissue extracts; fresh or cultured tissues; apoptotic cells; subcellular components, such as membrane, cytoplasm, and nuclear fractions from cells and tissues; purified proteins; partially purified proteins; laser captured tissue; or paraffin embedded and fixed tissue. In a preferred embodiment, the tissue comprises subject-derived tumor tissue.

In other aspects of the invention, the antibody-producing cells may be obtained from a transgenic mouse having antibody-producing cells that are capable of being immortalized without forming hybridomas. The transgenic mouse may comprise the genetic complement for producing human antibodies. Antibody-producing cells of the invention may be comprised in a mouse, and the selected antigen is administered to the mouse in a manner effective to induce the antibody-producing cells to produce antibodies. In certain aspects, the methods include contacting an antibody-producing cell with the desired antigen by co-culturing the antibody-producing cell with an antigen presenting cell, preferably the antigen presenting cell is a dendritic cell. In various embodiments, the antibody-producing cell comprises the genetic complement for human antibody production and produces human antibodies. The methods may further comprise purifying antibodies produced by said antibody-producing cells. In certain aspects, the methods may further comprise administering said antibodies to a subject in need of therapeutic antibodies.

Further embodiments of the invention include methods for generating an antibody-producing cell that produces a human antibody to a desired antigen. The methods may include obtaining an antibody-producing cell that conditionally expresses a transforming oncogene or expresses a conditionally functional transforming oncogene and expresses the genetic complement for human antibody production. Immortalization of the antibody-producing cell is typically effected by inducing the expression or function of said transforming oncogene. The methods may also include contacting the antibody-producing cells with a desired antigen in a manner effective to induce the cells to produce human antibodies against the antigen, wherein the antibody-producing cells are capable of being immortalized without forming hybridomas and immortalizing the antibody-producing cell. The conditionally functional transforming oncogene may be a temperature sensitive SV40 Large Tumor antigen (tsSV40Tag), preferably the tsSV40Tag is an A58S-SV40Tag. Aspects of the invention include induction of the expression or functionality of the transforming oncogene by culturing the antibody producing cells at temperatures from 25° C. to 35° C., preferably 30° C. to 35° C., more preferably at 33° C.

The methods may also include selecting and culturing single cells to produce a monoclonal population that produce monoclonal antibodies. In other aspects, the methods may include the selection and culture of multiple cells to produce a polyclonal population that produce polyclonal antibodies. In certain aspects, an antibody-producing cell includes a spleen cell(s). An antigen may include one or more peptides; proteins; glycoproteins; lipoproteins; carbohydrates; viruses; bacteria; pathogenic microorganisms; tissue; whole cells; biopsy tissue; patient-derived cells; tissue extracts; fresh or cultured tissues; apoptotic cells; subcellular components, such as membrane, cytoplasm, and nuclear fractions from cells and tissues; purified proteins; partially purified proteins; laser captured tissue; or paraffin embedded and fixed tissue. A tissue may comprise a subject-derived tumor tissue. In still further embodiments, antibody-producing cells are obtained from a transgenic mouse having antibody-producing cells that are capable of being immortalized without forming hybridomas. The antibody-producing cells may be comprised in a mouse, and the selected antigen is administered to the mouse in a manner effective to induce the antibody-producing cells to produce antibodies. In other aspects, the methods may further comprise purifying antibodies produced by the antibody-producing cell. The antibodies may be administered to a subject in need of therapeutic antibodies.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternative are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A and 1B illustrate an exemplary method for generating an in vitro, antibody producing immortalized splenocyte population against (FIG. 1A) pIII purified protein (5 μg/well) or (FIG. 1B) phage particle Fd-Tet (10¹¹ TU/well).

FIG. 2 illustrates an exemplary method for immortalizing dendritic cells and FACS analysis of the immortalized dendritic cells using anti-murine antibodies-CD80, -CD86, and -H2k.

FIG. 3A-3C illustrates an exemplary image of the morphology of immature immortal bone marrow derived cells (dendritic cells, DC).

FIG. 4A-4C represent an exemplary method for generating and assaying for anti-tumor antibodies. FIG. 4A and FIG. 4B represent an ELISA of 3×10⁴ exponentially growing KS cells and FIG. 4C represents an ELISA of 1.5×10⁴ MSC. Antibodies were plated directly from culture supernatants. Polyclonal serum was used as a positive control in FIG. 4B and FIG. 4C. The reaction was developed with OPD and absorbance was read at 450 nm.

FIG. 5 shows exemplary morphology of a culture of splenocytes from an immunized mouse after two months in culture. Follicular dendritic cells, clones of plasmocytes (producing antibodies B cells), macrophages and still unidentified epithelial-like cells (probably reticular epithelial cells) are observed.

FIGS. 6A and 6B represent an exemplary method for generating immortalized populations of and screening for thymus cells. FACS analysis of two cell surface protein populations is shown with FIG. 6A showing CD3 staining and FIG. 6B showing H2K staining.

FIG. 7 shows an exemplary time course for introducing an antigen to an immortalized cell population in culture.

FIG. 8 shows the results of an exemplary screening and validation of antibodies produced from immortalized spleen cells specifically antibodies obtained from H-2K^{b}-tsA58 transgenic mouse-derived immortal splenocytes exposed to filamentous phage (fd-tet) 810 or recombinant phage capsid pIII 820.

FIG. 9 shows the results of an exemplary screening and validation of antibodies produced from immortalized spleen cells, specifically antibodies obtained from H-2K^{b}-tsA58 transgenic mouse-derived immortal splenocytes exposed to filamentous phage (fd-tet) 910. Clones 1-3 correspond to clones that underwent freeze/thaw. Clones 4-9 correspond to different wells expanded from 96-well plates to 24-well plates and cultured for 6 weeks; Clone 10 indicates cultured medium alone as a negative control. Other controls included were pre- and post-immune sera.

FIG. 10 shows an evaluation of antibodies produced from immortalized spleen cells, specifically antibodies obtained from H-2K^{b}-tsA58 transgenic mouse-derived immortal splenocytes exposed to filamentous phage (fd-tet) 1010, recombinant phage capsid pIII 1020, or Bovine Serum Albumin (BSA) 1030. Clone 1, culture medium, negative control; clone 2, pre-immune serum; clones 3-7 correspond to supernatants derived different monoclonal lines after 8 weeks in culture. Bars correspond to the mean. Standard errors of the mean were less than 1% of the mean.

FIG. 11 shows a western blot analysis of the reactivity of supernatants from H-2K^{b}-tsA58 transgenic mouse-derived immortal splenocytes producing antibodies against phage proteins. Reactivity was evaluated after incubation with pre-immune serum 1110, post-immune serum 1120, an anti-phage antibody 1130, or supernatants containing anti-phage IgGs secreted from immortal splenocyte clones 1140, as indicated. Cell culture media alone 1150 served as an additional negative control. Antibodies reacting specifically against pIII 1160 and pVIII phage capsid proteins 1170 (arrows) were detected in supernatants from H-2K^{b}-tsA58 transgenic mouse-derived immortal splenocytes.

### DESCRIPTION OF ILLUSTRATIVE EMBODIMENTS

Monoclonal antibody production typically requires immortalization of splenocytes by somatic fusion to a myeloma cell line partner (hybridoma formation). Although hybridomas can be immortal, they may depend on a feeder cell layer and may lack genetic stability. Since the inception of hybridoma technology, efforts to improve efficiency and stability of monoclonal antibody-producing cell lines have not brought about substantial progress. Moreover, suitable human multiple myeloma-derived cell lines for the production of human antibodies have been very difficult to develop. The inventors describe a strategy that greatly simplifies the generation of antibodies and eliminates the need for hybridomas.

In certain embodiments, antibody producing cells, e.g., splenocytes, or antigen presenting cells, e.g., dendritic cells, are derived from transgenic mice harboring a polynucleotide encoding a mutant temperature-sensitive oncogene whose expression is under the control of an appropriate promoter, which allows a cell containing the polynucleotide to be conditionally immortalized at permissive temperatures. In preferred embodiments, the temperature sensitive oncogene is a simian virus 40 large tumor antigen (tsSV40Tag) under the control of a mouse major histocompatibility promoter. These splenocytes are immortalized at permissive temperatures (e.g., 33° C.) and produce antibodies without having to form hybridomas. This approach may be used for generation and production of both polyclonal and monoclonal antibodies. The growth properties and stability of these hybridoma free cells provide for additional compositions and methods for high-throughput discovery and antibody-based immunotherapy.

Further embodiments of the invention include processes, compositions, and methods for the generation and use of hybridoma-free antibodies, i.e., antibodies produced without the formation of a hybridoma. One embodiment of the invention includes compositions and methods for generation of hybridoma-free murine monoclonal or polyclonal antibodies.

In further embodiments, the methods may include contacting an appropriate cell type expressing a temperature sensitive oncogene, e.g., simian virus 40 large tumor antigen (tsSV40Tag), in vitro, ex vivo, or in vivo with an antigen to produce an antibody. In certain aspects, antibody producing cells, preferably splenocytes, are isolated and immortalized from a mouse expressing tsSV40Tag. In one such mouse, the ImmortoMouse®, a H-2K^{b}-tsA58 transgenic mouse, expression of the nucleic acid encoding a tsSV40Tag is under the control of the major histocompatibility promoter (Jat et al., 1991, incorporated herein by reference in its entirety). Cells derived from an ImmortoMouse® remain immortal if cultured at 33° C. (Jat et al., 1991). Various methods and compositions comprising transgenic mice and cells derived from transgenic mice expressing tsSV40Tag are described in the patent literature, for examples see U.S. Pat. Nos. 6,399,384; 5,866,759; 5,688,692; and 5,270,191, each of which is incorporated herein in its entirety. In other aspects, antibody producing or antigen presenting cells may be isolated and cultured from various tissues of a tsSV40Tag expressing animal including, but not limited to bone marrow, thymus, brain, or reproductive tissue. In still other aspects, stem cells may be isolated and cultured from such tissue samples.

In still further embodiments, harvested cells (e.g., from an ImmortoMouse®) may be contacted with one or more antigen(s) followed by evaluation of production of antibody against the antigen, including assessment of the specificity of antigen binding. Various sub-populations of the cells may be made or cloned and stored for future use.

In yet still further embodiments of the invention include breeding a mouse harboring a conditionally expressed or functional transforming oncogene with a transgenic mouse comprising the genetic complement for human antibody production. In one embodiment, cells from a mouse harboring both a conditionally expressed or functional transforming oncogene and genetic complement for human antibody production may be harvested (e.g., splenocytes, thymocytes, B cells) creating an immortalized cell populations that produces human antibodies. There are various patents that describe compositions and methods for using mice in the production of human or xenogeneic antibodies, for examples of this technology see U.S. Pat. Nos. 6,673,986; 6,657,103; 6,162,963; 6,235,883; 6,150,584; 6,114,598; 6,075,181 and 5,939,598, each of which is incorporated herein by reference in its entirety.

I. Antibody Production

Typically, antibodies of the invention are produced by immunizing mice, or other animals having cells that are conditionally immortalizable, or contacting an immortalized or potentially immortalizable cell with an antigen of interest. Immortal or potentially immortal cells may express a transforming gene that is conditionally functional, e.g., only functional at or below a certain temperature or only expressed under particular growth conditions. For example, a thermolabile large T antigen (tsSV40Tag) that is encoded by the simian virus 40 early-region mutant tsA58 may be used to establish transgenic mice or immoratlizable cell lines. These cell lines may grow continuously at permissive temperature (e.g., 33° C.) or in a particular environment, (e.g., in the presence of tetracycline), but upon shift-up to the non-permissive temperature (37-39.5° C.) or the removal or addition of a regulatory factor show arrested cell growth. The growth arrest occurs in either the G1 or G2 phase of the cell cycle. After growth arrest, the cells remain metabolically active as assayed by general protein synthesis and the ability to exclude trypan blue. These cell lines cannot divide at the non-permissive temperatures or conditions.

Following immunization of a transgenic animal, somatic cells with the potential for producing antibodies or presenting antigens, specifically B lymphocytes (B cells) or dendritic cells (DC), respectively, are selected for use in the monoclonal antibody generating protocol. These cells may be obtained from biopsied spleens, tonsils, lymph nodes, or peripheral blood samples from one or more subjects, including but not limited to mice, rats, rabbits, dogs cats, goats, cows, horses, sheep or humans. Spleen cells and peripheral blood cells are preferred, the former because they are a much richer source of antibody-producing cells that are in the dividing plasmablast stage, and the latter because peripheral blood is easily accessible. Often, a panel of animals will have been immunized and the spleen of the animal with the highest antibody titer will be removed and the lymphocytes of the spleen obtained by homogenizing the spleen with a syringe. Typically, a spleen from an immunized mouse contains approximately 5×10⁷ to 2×10⁸ lymphocytes.

An animal expressing a transforming protein is immunized (e.g., H-2Kb-tsA58 ImmortoMouse®) or cells expressing a transforming protein are exposed to an antigen (e.g., filamentous fd-tet phage (Zacher et al., 1980)) in order to induce production of an antibody that binds an antigen of interest. The immunization or contact may be repeated one or more times over various periods of time, for example immunization or antigen exposure may be once every 1, 2, 3, 4, 5, 6, 7, 8, 9, 10, 11, 12 or more days or weeks. In certain aspects immunization or antigen exposure may be every other day or week, or every third, fourth, fifth or more day or week. Immunization or antigen exposure may be carried out over 2, 3, 4, 5, 6 7, 8, 9, 10, 11, 12, 13, 14, 15, or more weeks and even months, preferably for about 12 weeks. An antigen preparation is administered by one or more routes, including intravenous (i.v.), intraperitoneal (i.p.), intradermal, subcutaneous (s.c.) or various combinations thereof. Animals may be bled after each boost and ELISA used to monitor anti-antigen antibody titers in the serum.

Organs from an immunized animals may be harvested or biopsied (e.g., spleen), or antigen exposed cells harvested and placed in a cell culture medium. Cells are typically released from an organ by gentle pressure applied to the capsule of the organ, which is placed between two frosty glass slides. Next, antibody producing cells (e.g., splenocytes) are resuspended in an appropriate growth medium, preferably a hybridoma medium, and grown at low-density. Tissue debris are gravity-cleared by serially transferring of cells to fresh containers. Typically, a total of about 2×10⁸ cells are distributed in 6-, 24-, and 96-well plates and cultured at 33° C. The culture medium is changed completely at least 2, 3, 4, or more times during 2-3 weeks. Clones are typically observed in greater than 90% of the wells after 3 weeks. The plates are monitored, and fresh medium added to each well every 3 weeks or so. Positive wells may be subcloned by limiting dilution (Harlow and Lane, 1988); some of the clones are also expanded to 24-well and 96-well plates to monitor reactivity after long term culture. Splenocyte “clumping” may be avoided by carefully suspending the cells in each well and by using serum-free medium. Typically, after counting and plating the suspensions at about 0.1-0.5 cells per well, each 96-well plate is systematically inspected under the microscope.

After a population of antibody producing cells have been isolated they are screened and selected for an antibody with a characteristic of interest, for example, selective binding affinity for a particular pathogen or protein. Once a subset of cells has been selected the cell or cells identified are cloned and propagated. ELISA against the antigen of interest, e.g., a filamentous phage and/or recombinant protein, is typically use to screen and select cells producing antibodies of interest, exemplary methods are described below and in Harlow and Lane (1988). Negative controls may include BSA, hybridoma medium alone, pre-immune serum, and secondary antibody for comparison with a cell isolated from an immunized animal or exposed to an antigen. Immune polyclonal serum and anti-antigen antibody may serve as positive controls. In particular aspects, antibodies are plated directly from culture supernatants. Cells from the positive wells are subcloned by limiting dilution to obtain monoclonal lines. Subclones emerging from these procedures are tested against various antigens by ELISA. Reactivity is monitored in an ELISA reader.

Once a cell producing a promising antibody is identified and subcloned, western blot analysis, as well as other antigen binding assays, are performed to confirm and further characterize the resulting antibody. Typically, antigens are resolved using SDS/PAGE electrophoresis and electrotransferred to polyvinylidene fluoride membrane (Bio-Rad). The membrane may be divided into strips, blocked by 5% nonfat milk in PBS, followed by washing in an appropriate wash buffer, e.g., PBS containing 0.1% Tween 20. Strips are incubated with preimmune serum (1:1,000), postimmune serum (1:1,000), positive control antibodies, supernatants containing IgGs secreted from immortal cell clones, or cell culture media. After various washes, a detection agent-conjugated to secondary antibody (peroxidase-conjugated secondary Ab) (Bio-Rad) is added to the strips and incubated at room temperature. Strips are washed, and the reactivity is detected, for example by enhanced chemiluminescence (ECL) (Amersham Biosciences, Piscataway, N.J.).

Mice are typically given intraperitoneal (i.p.) injections of antigen at 2-week intervals over a period of 2 months. After the third and fourth immunization, the serum of each immunized mouse may be analyzed by an ELISA assay. Typically, the spleen from the mouse or mice with the highest anti-antigen antibody titer is removed, and splenocytes are isolated. Single clones may be obtained by limiting dilution. Antibody production is monitored by ELISA on cell culture supernatants using the initial antigen (Harlow and Lane, 1988).

Culturing provides a population of immortalized cells from which specific subclones are selected. Typically, selection of immortalized cells is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like. General methods for preparing and characterizing antibodies are well known in the art (See, e.g., Antibodies: A Laboratory Manual, Cold Spring Harbor Laboratory, 1988; incorporated herein by reference).

One of the criteria for successful generation of a therapeutic protein from a cell is to obtain a cell line that maintains stability of production. If this is not achieved, it can generate problems for process yields, effective use of time and money, and for regulatory approval of products. There are several studies that have reported on the instability of protein production from hybridoma cell lines. The cause of instability of protein production in hybridomas are varied and, in many cases, the exact molecular mechanisms are still unknown.

With respect to polyclonal antibody production, antibodies may be generated ex vivo and may eliminate the need for multiple antigen injections and bleedings in a target antibody-producing animal such as a mouse or rabbit. This technology allows one to produce polyclonal population of antibodies, T cells, or natural killer cells, primed and expanded based on exposure to a target antigen or group of antigens associated to a given tissue, cell population, or protein. Such a process is more difficult with hybridomas because of the dominance of certain clones over others.

As used herein, the term “antibody” refers to any antibody-like molecule that has an antigen binding region, and includes polyclonal and monoclonal antibodies, as well as antibody fragments such as Fab′, Fab, F(ab′)₂, single domain antibodies (DABs), Fv, scFv (single chain Fv), and the like. Techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

In certain embodiments of the present invention, immortalized splenocytes derived from transgenic mice harboring a mutant temperature-sensitive (ts) simian virus 40 (SV40) large tumor antigen (Tag) under the control of a mouse major histocompatibility promoter (named H-2K^{b}-tsA58 transgenic mouse; ImmortoMouse®) are immortal and alleviate the need for hybridoma production.

Once an antibody has been identified using the described methods, the appropriate gene or genes may be amplified, cloned and transfected into another cell for production of antibodies. For examples see U.S. Pat. Nos. 5,658,570, 6,165,745, and 6,602,503 as well as Daniell, 2001; Breitling and Dubel, 1999, each of which is incorporated herein by reference.

II. Transgenic Mice

Studies on cell lines have greatly improved our understanding of many important biological questions. Generation of cell lines is facilitated by the introduction of immortalizing oncogenes into cell types of interest (Jat et al., 1991). One gene known to immortalize many different cell types in vitro encodes the SV40 large tumor antigen (SV40TAg). To circumvent the need for gene insertion in vitro to generate cell lines, transgenic mice harboring the SV40TAg gene have been created (Jat et al., 1991). Since previous studies have shown that SV40TAg expression in transgenic mice is associated with tumorigenesis and aberrant development, a thermolabile SV40Tag, tsA58 (tsSV40Tag), that is temperature sensitive for transformation, was used to reduce the levels of functional SV40TAg present in vivo under typical conditions found in a whole organism.

To direct expression to a broad range of tissues a mouse major histocompatibility complex H-2Kb promoter that is both widely active and can be induced by interferons was used. The tsSV40TAg mRNA was expressed in tissues of all animals harboring the hybrid construct. Development of all tissues was macroscopically normal. One strain of H-2Kb-tsA58 mice has been bred through several generations to homozygosity and transmits a functional copy of the transgene. These mice are termed “ImmortoMice.” The ImmortoMouse® is commercially available from Charles River Labs, Wilmington, Mass. Many different types of conditionally immortal cell lines have been derived from ImmortoMouse® but, this well established mouse model has not been exploited for the generation of monoclonal antibody-producing cells.

ImmortoMouse® was developed for its ability to generate expanded populations of individual cell types able to undergo normal differentiation in vitro and in vivo for use in the investigation of the cellular mechanisms of differentiation and for cell transplantation studies related to tissue repair. The H-2Kb-tsA58 mouse allows the direct derivation of conditionally immortal cell lines from a variety of tissues by the growth of isolated cells under appropriate conditions. In these mice the tsSV40Tag is controlled by the interferon-inducible Class I antigen promoter. Cells can be grown for extended periods in vitro by growing them at 33° C. in the presence or absence of interferon, while still retaining the capacity to undergo normal differentiation in vivo and in vitro.

III. Human Antibody Production

In one embodiment of the invention, transgenic mice comprising a conditionally functional transforming oncogene, e.g., an ImmortoMouse®, may be crossed with transgenic mice harboring genes from other species encoding various genetic components for antibody production (as detailed below) to generate cell lines producing antibodies of the other species, such as human antibodies.

The ability to produce a diverse repertoire of fully human monoclonal antibodies has applications in human therapy. One of the most promising approaches to the production of therapeutic human polyclonal or monoclonal antibodies is the creation of a mouse strain engineered to produce a large repertoire of human antibodies in the absence of mouse or other non-human antibodies (e.g., XenoMouse®). Recently, mice have been generated by introducing segments of human immunoglobulin loci into the germline of mice deficient in mouse antibody production as a result of gene targeting. These mice produce significant levels of fully human antibodies with a diverse adult-like repertoire and, upon immunization with antigens, generate antigen-specific human antibodies. The XenoMouse® is equipped with approximately 80% of the human heavy chain antibody genes and a significant amount of the human light chain genes. The complex assembly of these genes together with their semi-random pairing allows the mouse to recognize a diverse repertoire of antigen structures. In addition, the mouse is capable of processing extremely high affinity, completely human antibodies. There are multiple strains of XenoMouse® animals available. Each strain is capable of producing a different class of antibody for various applications. Such strains of mice may provide the optimal source for producing human antibodies with high affinity and specificity against a broad spectrum of antigens, including human antigens.

The XenoMouse® generates antibodies with fully human protein sequences using genetically engineered strains of mice in which mouse antibody gene expression is suppressed and functionally replaced with human antibody gene expression, while leaving intact the rest of the mouse immune system. By introducing human antibody genes into the mouse genome, transgenic mice with such traits can be bred indefinitely. Importantly, these transgenic mice are capable of generating human antibodies to human antigens because the only human products expressed in the mice (and therefore recognized as “self”) are the antibodies themselves. All the other machinery is mouse machinery, thus any other human tissue or protein is recognized as foreign by the mouse and an immune response will be mounted.

Abnormal synthesis of some human proteins, for example cytokines hormones and growth factors or their receptors, contribute to various human diseases. Regulating these proteins by neutralization or total elimination using human antibodies may be used to treat or completely eliminate the disease. The ability of these transgenic mice to generate cells that may be used in production of human antibodies against human antigens could offer an advantage in the treatment, diagnosis, or cure of various disease states. One challenge has been to produce enough of a human antibody against a given antigen in a stable cell line. This problem may be solved by embodiments of this invention. In one embodiment, a cross-bred mouse population (e.g., ImmortoMouse®/Xenomouse® cross) may produce immortalized splenocytes capable of producing antibodies against any human antigen without the need to produce hybridomas.

The Xenomouse, or animals with similar genetic modifications, generate antibodies with 100% human protein sequences that differ from chimeric and other humanization technologies. Other advantages of using these mice are that the antibodies produced using XenoMouse® technology may be expected to offer a better safety profile and to be eliminated less quickly from the human body, reducing the frequency of dosing.

XenoMouse® technology uses the natural in vivo affinity maturation process to generate antibody product candidates usually in two to four months. These antibody product candidates may have affinities as much as a hundred to a thousand times higher than those seen in phage display. In contrast to antibodies generated using humanization and phage display technology there is no need for any subsequent engineering, a process that at times has proven to be challenging and time consuming. Therefore, an antibody's structure may remain intact from the initial antibody selected to the final commercial antibody.

In the past, once an antibody with the desired characteristics has been identified, pre-clinical material can be produced either directly from hybridomas or from recombinant cell lines. In addition to potential timesaving, hybridoma-free production avoids the need to produce antibodies in hybridomas or recombinant cell lines. Thus, embodiments of this invention may satisfy a need for producing human antibodies in long-term culture without the need for hybridomas.

Typically, mouse-generated monoclonal antibodies are rejected by patients whose immune systems recognized them as foreign because they are not human proteins. The patients often produce a human anti-mouse antibody, or HAMA. This response reduces the effectiveness of the antibody by neutralizing the binding activity. Any subsequent administrations of mouse antibodies may also prove toxic. Using the methods described herein, antibodies to almost any medically relevant antigen, human or otherwise may be generated. The ability to produce multiple antibodies to choose from may be important in selecting the optimal antibody product. In one embodiment of the invention, an immortalized population of human monoclonal antibody-producing splenocytes may be produced by the disclosed methods.

In addition, Medarex (Princeton, N.J.) has developed a system called the UltiMAb Human Antibody Development System^{SM}. This system has created various types of fully human (100% human protein sequences) antibodies. These mice contain genes encoding human antibodies. These monoclonal antibodies are more likely to have favorable safety profiles and be eliminated less rapidly from the human body, potentially reducing the frequency and amount of dosing required to affect disease targets. These mice may also be used in combination with the hybridoma-free antibody production methods described herein.

In brief, the methods described herein may produce antibodies against tumor antigens or proteins expressed in a variety of disease states (e.g., inflammation) for therapeutic or diagnostic purposes. One embodiment may use human monoclonal antibodies to target or enhance delivery drugs (e.g., cytotoxins) to a target cell population due to the affinity and selectivity of the antibody for a target cell population, e.g., tumor cells. On the other hand, human monoclonal antibodies may be used to inhibit the function of a receptor (for example a growth factor receptor such as the epidermal growth factor receptor (EGFR)) which is over-expressed in many disease states and various types of tumors. EGFR signaling can be blocked leading to cell death or inhibition of proliferation. Other applications include imaging of tumors with fluorescent antibodies that bind to a target cell population, e.g., tumor cells.

In certain embodiments, antibodies manufactured by the methods described herein may be used to identify or target vascular zip codes identified using phage display technology, described below. Vascular zip codes are specific and unique addresses in the human body to which drugs may be more efficiently and effectively delivered, for examples see U.S. Pat. Nos. 5,622,699, 6,174,687 and 6,232,287, each of which is incorporated herein by reference. Vascular targeting may improve, for example, the effectiveness of a therapy by zeroing it in on the tumor site while sparing the healthy parts of the body. By administering a collection of more than a billion peptide sequences displayed in microscopic particles called phage, the peptides home preferentially to specific areas of the body. This large-scale screening shows that the tissue distribution of circulating peptides is non-random and that certain peptides direct and bind to different organs.

These peptides typically bind to receptors present in the tissues and blood vessels of organs. While traveling through the body, peptides may simulate the behavior of ligands (peptide-binding proteins) and interact with cellular receptors in the cells, tissues, blood vessels or organs of a subject. Ligands may be identified by screening the peptide libraries in vivo and in turn the ligands can be used to identify a receptor. Then human monoclonal antibodies may be made to the receptor using the methods described herein. Another application may be to identify targets by identifying the circulating repertoire of antibodies in patients (Mintz et al., 2003) and then generate human antibodies to these targets in order to develop targeted therapies or passive immunization in certain cases.

Historically it takes a new pharmaceutical product nearly six years of pre-clinical development before human clinical trials are initiated. Using fully human monoclonal antibody technology, it is possible to reduce that time to less than two years. Moreover, the costs of development are likely to be a fraction of those associated with the chemical compounds traditionally developed by the pharmaceutical industry.

IV. Antigens for Antibody Production

Embodiments of the invention include immunizing transgenic mice or exposing cells expressing a conditionally functional transforming gene with various antigens to produce stable lines of antibody producing or antigen presenting cells. Antigens within the scope of the invention may include any molecule or macromolecular assemblage that is capable of provoking a humoral and cellular immune response in a subject, including but not limited to peptides, proteins, glycoproteins, lipoproteins, viruses, bacteria, pathogenic microorganisms and diseased human cells. It is important to note that in certain embodiments, the use of ex vivo methods for generation of antibodies (i.e., antibody generation outside of the subject to be treated), allows one to generate antibodies to an antigen that may not be generated within a mammal, such as a human. Generation of antibodies by the inventive methods allows one to bypass the effects of tolerance within the subject. Thus, an expanded variety of antibodies may be generated. These antibodies may allow one to target antigens that were previously difficult to target.

In certain embodiments, it may be desirable to make antibodies (e.g., monoclonal and/or polyclonal) against the identified targeting peptides (e.g., peptides that target specific organs) or their receptors or even whole cells such as tumor cells. The appropriate targeting peptide or receptor, or portions thereof, may be coupled, bonded, bound, conjugated, or chemically-linked to one or more agents, including adjuvants, via linkers, polylinkers, or derivatized amino acids. In certain aspects adjuvants include the use of colloidal gold (Dykman et al., 1996, which is incorporated herein by reference in its entirety). This may be performed such that a bispecific or multivalent composition or vaccine is produced. It is further envisioned that the methods used in the preparation of these compositions are familiar to those of skill in the art and should be suitable for administration to human subjects, i.e., pharmaceutically acceptable. Preferred agents include carriers such as keyhole limpet hemocyanin (KLH) or bovine serum albumin (BSA). In various embodiments, subjects may be any higher vertebrate, including but not limited to mice, rabbits, chickens, goats, sheep, cows, dogs and humans.

In certain embodiments, anti-idiotypic antibodies or antibodies to receptors of a targeting peptide may be produced. A “targeting peptide” is a peptide comprising a contiguous sequence of amino acids, that is characterized by selective localization to a subject organ or tissue. Selective localization may be determined, for example, by methods disclosed below, wherein the putative targeting peptide sequence is incorporated into a protein that is displayed on the outer surface of a phage. Administration to a subject of a library of such phage that have been genetically engineered to express a multitude of such targeting peptides of different amino acid sequence is followed by collection of one or more organs or tissues from the subject and identification of phage found in that organ or tissue. A phage expressing a targeting peptide sequence is considered to be selectively localized to a tissue or organ if it exhibits greater binding in that tissue or organ compared to a control tissue or organ.

In general, selective localization of a targeting peptide should result in at least a two-fold enrichment of the phage in the target organ or tissue, compared to a control organ or tissue. Selective localization resulting in at least a three-fold, four-fold, five-fold, six-fold, seven-fold, eight-fold, nine-fold, ten-fold or higher enrichment in the target organ compared to a control organ or tissue is preferred. Alternatively, a phage expressing a targeting peptide sequence that exhibits selective localization should show an increased enrichment in the target organ compared to a control organ when phage recovered from the target organ are re-injected into a second host for another round of screening. Another alternative means to determine selective localization is that phage expressing the putative target peptide exhibit at least a two-fold, more preferably at least a three-fold enrichment in the target organ compared to control phage that express a non-specific peptide or that have not been genetically engineered to express any putative target peptides. Another means to determine selective localization is that localization to the target organ of phage expressing the target peptide is at least partially blocked by the co-administration of a synthetic peptide containing the target peptide sequence. “Targeting peptide” and “homing peptide” are used synonymously herein.

Other antigens for antibody production may include samples from biopsies, patient-derived cells, patient-derived fresh tumor tissue, tissue extracts, fresh or cultured tissues. It is important to include tissue components and not just cells because some antigens of relevance may be in an extracellular component and/or in the stroma. Other antigens for antibody generation may include but are not limited to apoptotic cells, membrane components, cytoplasm, nuclear fractions from cells and tissues, purified proteins, partially-purified proteins, laser captured tissue, paraffin embedded or fixed tissue.

A. Phage Display

Antigens or antigenic candidates may be identified using phage display. The methods may include the in vivo administration of phage display libraries. In various embodiments of the invention, ligands may be identified and then used for further identification of receptors to these ligands and then the receptors may be used to generate monoclonal antibody producing immortalized splenocytes. Various methods of phage display and methods for producing diverse populations of peptides are well known in the art. For example, see U.S. Pat. Nos. 5,223,409, 5,622,699 and 6,068,829, each of which is incorporated herein by reference and describe methods for preparing a phage library.

The phage display technique involves genetically manipulating bacteriophage so that small peptides can be expressed on their surface (Smith et al., 1985, 1993). The potential range of applications for this technique is quite broad, and the past decade has seen considerable progress in the construction of phage-displayed peptide libraries and in the development of screening methods in which the libraries are used to isolate peptide ligands. For example, the use of peptide libraries has made it possible to characterize interacting sites and receptor-ligand binding motifs within many proteins, such as antibodies involved in inflammatory reactions or integrins that mediate cellular adherence. This method has also been used to identify novel peptide ligands that serve as leads to the development of peptidomimetic drugs or imaging agents (Arap et al., 1998a).

The most efficient amino acid sequences for targeting a given organ or tissue can be isolated by “biopanning” (Pasqualini and Ruoslahti, 1996; Pasqualini, 1999). In brief, a library of phage containing putative targeting peptides may be administered or put in contact with a cell population (e.g., splenocytes), an animal or human subject and cell extracts or samples of organs or tissues containing phage may be collected. In one embodiment utilizing filamentous phage, the phage may be propagated in vitro between rounds of biopanning in pilus-positive bacteria. The bacteria are not lysed by the phage but rather secrete multiple copies of phage that display a particular insert. Phage that bind to a target molecule can be eluted from the target organ or tissue and then amplified by growing them in host bacteria. If desired, the amplified phage can be administered to a human host and samples of organs or tissues again collected. Multiple rounds of biopanning can be performed until a population of selective binders is obtained. The amino acid sequence of the peptides is determined by sequencing the DNA corresponding to the targeting peptide insert in the phage genome. The identified targeting peptide can then be produced as a synthetic peptide by standard protein chemistry techniques (Arap et al., 1998a, Smith et al., 1985). This approach allows circulating targeting peptides to be detected in an unbiased functional assay, without any preconceived notions about the nature of their target.

Once a candidate target is identified as the receptor of a targeting peptide, it can be isolated, purified and cloned by using standard biochemical methods (Pasqualini, 1999; Rajotte and Ruoslahti, 1999). These purified proteins may then be used as an antigen for immunization or exposure of a cell population such as splenocytes from an ImmortoMouse®, an ImmortoMouse® cross producing humanized cell populations or other conditionally immortalizable cell lines, such as monoclonal antibody producing splenocytes. Then these antibody-producing cells may be used to generate specific antibody populations against the targeted receptor or antigen.

Previous in vivo selection studies performed in mice preferentially employed libraries of random peptides expressed as fusion proteins with the gene III capsule protein in the fuSE5 vector (Pasqualini and Ruoslahti, 1996). The number and diversity of individual clones present in a given library is a significant factor for the success of in vivo selection. It is preferred to use primary libraries, which are less likely to have an over-representation of defective phage clones (Koivunen et al., 1999). The preparation of a library should be optimized to between 10⁸-10⁹ transducing units (T.U.)/ml. In certain embodiments, a bulk amplification strategy is applied between each round of selection.

Phage libraries displaying linear, cyclic, or double cyclic peptides may be used within the scope of the invention. However, phage libraries displaying 3 to 10 random residues in a cyclic insert (CX₃₋₁₀C) are preferred, since single cyclic peptides tend to have a higher affinity for the target organ than linear peptides. Libraries displaying double-cyclic peptides (such as CX₃C X₃C X₃C; Rojotte et al., 1998) have been successfully used. However, the production of the cognate synthetic peptides, although possible, can be complex due to the multiple conformers with different disulfide bridge arrangements.

V. Proteins and Peptides

In certain embodiments, antigen compositions may comprise at least one protein, peptide or peptide-like compound that may be used in antibody production. As used herein, a protein or peptide generally refers, but is not limited to, a protein of greater than about 200 amino acids, up to a full length sequence translated from a gene; a polypeptide of greater than about 100 amino acids; and/or a peptide of from about 3 to about 100 amino acids. For convenience, the terms “protein,” “polypeptide” and “peptide are used interchangeably herein. In certain embodiment, a protein is an antibody produced by the methods described herein.

In certain embodiments, the size of the at least one protein or peptide may comprise, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino acid residues.

As used herein, an “amino acid residue” refers to any naturally occurring amino acid, any amino acid derivative or any amino acid mimic known in the art. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties.

Accordingly, the term “protein or peptide” encompasses amino acid sequences comprising at least one of the 20 common amino acids found in naturally occurring proteins, or at least one modified or unusual amino acid.

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases (www.ncbi.nlm.nih.gov/). The coding regions for known genes may be amplified and/or expressed using the techniques disclosed herein or as would be know to those of ordinary skill in the art. Alternatively, various commercial preparations of proteins, polypeptides and peptides are known to those of skill in the art.

A. Peptide Mimetics

Another embodiment for the preparation of polypeptides according to the invention is the use of peptide mimetics for use as an antigen. Mimetics are peptide-containing molecules that mimic elements of protein secondary structure. See, for example, Johnson et al., 1993, incorporated herein by reference in its entirety. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used to engineer antigens having many of the natural properties of the targeting peptides disclosed herein, but with altered and even improved characteristics.

B. Fusion Proteins

Other embodiments of the invention concern using fusion proteins as antigen. These molecules generally have all or a substantial portion of a peptide of interest, linked at the N- or C-terminus, to all or a portion of a second polypeptide or protein. For example, fusions may employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. In preferred embodiments, the fusion proteins of the embodiments comprise a peptide linked to a antigenic protein or peptide to elicit an immune response.

In other embodiments, fusion proteins include antibodies produced by the inventive methods that may be fused with therapeutic peptides. Examples of proteins or peptides that may be incorporated into a fusion protein include cytostatic proteins, cytocidal proteins, pro-apoptosis agents, anti-angiogenic agents, hormones, cytokines, growth factors, peptide drugs, antibodies, Fab fragments antibodies, antigens, receptor proteins, enzymes, lectins, MHC proteins, cell adhesion proteins and binding proteins.

These examples are not meant to be limiting and it is contemplated that within the scope of the present invention virtually any protein or peptide could be incorporated into a fusion protein for use in the present invention. Methods of generating fusion proteins are well known to those of skill in the art. Such proteins can be produced, for example, by chemical attachment using bi-functional cross-linking reagents, by de novo synthesis of the complete fusion protein, or by attachment of a DNA sequence encoding a first peptide to a DNA sequence encoding a second peptide or protein, followed by expression of the intact fusion protein.

C. Protein Purification

In certain embodiments a protein (e.g., antibody) or peptide may be isolated or purified. Protein purification techniques are well known to those of skill in the art. These techniques involve, at one level, the homogenization and crude fractionation of the cells, tissue or organ to polypeptide and non-polypeptide fractions. The protein or polypeptide of interest may be further purified using chromatographic and electrophoretic techniques to achieve partial or complete purification (or purification to homogeneity). Analytical methods particularly suited to the preparation of a pure peptide are ion-exchange chromatography, gel exclusion chromatography, HPLC (high performance liquid chromatography), FPLC (AP Biotech), polyacrylamide gel electrophoresis, affinity chromatography, immunoaffinity chromatography and isoelectric focusing. An example of receptor protein purification by affinity chromatography is disclosed in U.S. Pat. No. 5,206,347, the entire text of which is incorporated herein by reference. One of the more efficient methods of purifying peptides is fast performance liquid chromatography (FPLC) or even HPLC.

A purified protein or peptide is intended to refer to a composition, isolatable from other components, wherein the protein or peptide is purified to any degree relative to its naturally-obtainable state. An isolated or purified protein or peptide, therefore, also refers to a protein or peptide free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the proteins in the composition.

Various methods for quantifying the degree of purification of the protein or peptide are known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “-fold purification number.” The actual units used to represent the amount of activity will, of course, be dependent upon the particular assay technique chosen to follow the purification, and whether or not the expressed protein or peptide exhibits a detectable activity.

Various techniques suitable for use in protein purification are well known to those of skill in the art. These include, for example, precipitation with ammonium sulfate, PEG, antibodies and the like, or by heat denaturation, followed by: centrifugation; chromatography steps such as ion exchange, gel filtration, reverse phase, hydroxylapatite and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

Affinity chromatography is a chromatographic procedure that relies on the specific affinity between a substance to be isolated and a molecule to which it can specifically bind to. This is a receptor-ligand type of interaction. The column material is synthesized by covalently coupling one of the binding partners to an insoluble matrix. The column material is then able to specifically adsorb the substance from the solution. Elution occurs by changing the conditions to those in which binding will not occur (e.g., altered pH, ionic strength, temperature, etc.). The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand.

D. Synthetic Peptides

Because of their relatively small size, some antigenic peptides can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, usually from about 6 up to about 35 to 50 amino acids, can be readily synthesized by such methods. Alternatively, recombinant DNA technology may be employed wherein a nucleotide sequence which encodes a peptide of the invention is inserted into an expression vector, transformed or transfected into an appropriate host cell, and cultivated under conditions suitable for expression.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Generation of Immortal Spleen Cells

A. Methods

Isolation of Splenocytes

Spleens from H-2K^{b}-tsA58 mice (Charles River Laboratories, Wilmington, Mass.) were collected in Dulbecco's modified Eagle's medium (DMEM). Cells were released by gentle pressure applied to the capsule of the organ, which was placed between two frosty glass slides. Red blood cells were lysed by using ammonium chloride and splenocytes were re-suspended in 15 ml of hybridoma medium with 10% CPSR plus hybridoma-enhancing supplements. Tissue debris were cleared by filtration through nylon mesh. The cells were distributed in 24-well plates (2×10⁶/well) and cultured at 33° C. The culture medium was replaced every other week.

Although other plate sizes may be used, it was found that the 24-well (1 ml of spleen suspension from 55 ml of spleen) may be preferred. The culture medium may be periodically replaced (e.g., every other week). Clones were observed in greater than 90% of the wells after 3 weeks.

Mouse Immunization

H-2K^{b}-tsA58 mice (Charles River Laboratories, Wilmington, Mass.) were immunized with filamentous fd-tet phage every other week for 12 weeks. In brief, a phage preparation containing 10⁷ transducing units (TU)/μl (total volume=1 ml) was administered by 4 routes (intravenously, intraperitoneally, intradermally, and subcutaneously). Mice were bled after each boost and ELISA was used to monitor anti-phage antibody titers in the serum. Animal experimentation involved standard established procedures reviewed and approved by the Institutional Animal Care and Use Committee from the University of Texas M. D. Anderson Cancer Center.

Screening and Generation of Clonal Antibody-Producing Splenocytes.

ELISA against filamentous phage and against recombinant phage capsid pIII protein was performed as previously described (Harlow and Lane, 1998). Bovine serum albumin (BSA), hybridoma medium alone, pre-immune serum and secondary antibody served as negative controls. Immune polyclonal serum and anti-phage antibody served as positive controls. Antibodies were plated directly from culture supernatants. Cells from the positive wells were sub-cloned by limiting dilution (0.1 or 0.5 cells per well in 96-well plates) in order to obtain monoclonal lines. Sub-clones emerging after two months were tested against the entire phage particle and the pill phage capsid protein by using ELISA. Reactivity was monitored in an ELISA reader.

Western Blot Analysis.

Filamentous fd-tet phage (10⁹ TU/lane) were boiled, resolved by a gradient 4-20% SDS-PAGE (Invitrogen Corp., Carlsbad, Calif.) and electrotransferred to Immuno-Blot polyvinylidene fluoride membrane (PVDF; Bio-Rad Laboratories, Inc., Hercules, Calif.). The membrane was divided into strips, blocked by 5% non-fat milk in phosphate-buffered saline (PBS) for 1 h at room temperature (RT) followed by a single wash with PBS containing 0.1% Tween 20 (PBS-T). Strips were incubated with pre-immune serum (1:1,000), post-immune serum (1:1,000), anti-fd-tet phage (Sigma-Aldrich, St. Louis, Mo.), supernatants containing anti-phage IgGs secreted from immortal splenocyte clones, or cell culture media alone for 2 h at RT. After three washes, a peroxidase-conjugated secondary antibody (Bio-Rad Laboratories, Inc., Hercules, Calif.) was added to the strips and incubated for 1 h at RT. Strips were washed three times and the reactivity was detected by enhanced chemiluminescence (ECL; Amersham Biosciences Corp., Piscataway, N.J.).

ELISA

The following is one example of an assay used to access the presence or absence of directed monoclonal antibody production. A selected antigen may be immobilized in PBS (10⁹ particles or 5 μg/well) on High Binding Assay Plates (Costar e.g., 24, 48 or 96-well plate). Control wells are coated with 2 mg bovine serum albumin (BSA) in PBS overnight at 4° C. Primary antibodies or control polyclonal species IgG (Sigma) are then incubated at a range of concentrations for 1 h at room temperature. The secondary antibody (anti-species-Fab alkaline phosphatase-conjugate, Sigma, 1:3000 in 3% BSA) is added and incubated for 1 h. The ELISA is developed with p-nitrophenyl phosphate (Sigma), and readings may be taken 1-4 h later at 405 nm (Reader 520, Organon Teknika).

Immunoprecipitation and Western Blot Analysis

An antigen of interest may be diluted in 50 mM Tris-HCl pH 7.6, 1% NP-40, 150 mM NaCl, and 0.1 mM ZnOAc in the presence of protease inhibitors. Protein concentration may be determined by the Lowry method (Bio-Rad). Proteins may be immunoprecipitated in the presence of the clones in question in the presence of protein G-sepharose (Pharmacia) at a concentration of around 5 μg/ml of monoclonal antibodies. Immunoprecipitated proteins may separated by SDS-PAGE, transferred to a nitrocellulose membrane, blotted with anti-monoclonal antibody (e.g., mouse or human) IgG HRP (Jackson Laboratories), and visualized by enhanced chemiluminescence (Renaissance, NEN). Alternatively, the protein of interest may be first separated by an SDS-PAGE gel then the proteins transferred to nitrocellulose paper and the probed with the monoclonal antibody population in question and visualize the results using with anti-monoclonal antibody (e.g., mouse or human) IgG HRP (Jackson Laboratories), and visualized by enhanced chemiluminescence (Renaissance, NEN).

Ex Vivo Immunization of Immortal Spleen Cultures

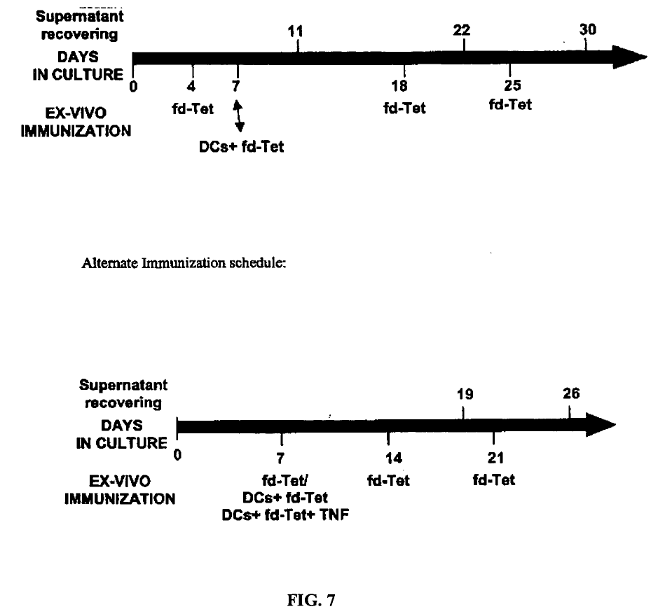

Approximately 5 days after plating of the cells recovered from the spleen, wells may receive an antigen dose for example phage (fd-Tet) in concentrations ranging from 0.5×10¹⁰ to 1×10¹² TU/2×10⁶ cells. This step may be considered a “priming step” or “first ex vivo immunization.” Further boosts may follow (e.g., the same amount of phage may be added) 18 and 25 days after priming (spleen I) 14 and 21 days after priming (spleenII). Alternatively, in a parallel experiment, splenocytes may be primed by co-incubation with dendritic cells (DC) previously exposed to phage (loaded with phage). In this example, there were no subsequent boosts. Ex vivo immunization may be performed as shown in the time line of FIG. 7. For ex vivo immunization with whole cells splenocytes where co-incubated with DCs loaded with apoptotic B16-F10 cells or apoptotic cells alone as above described.

B. Results

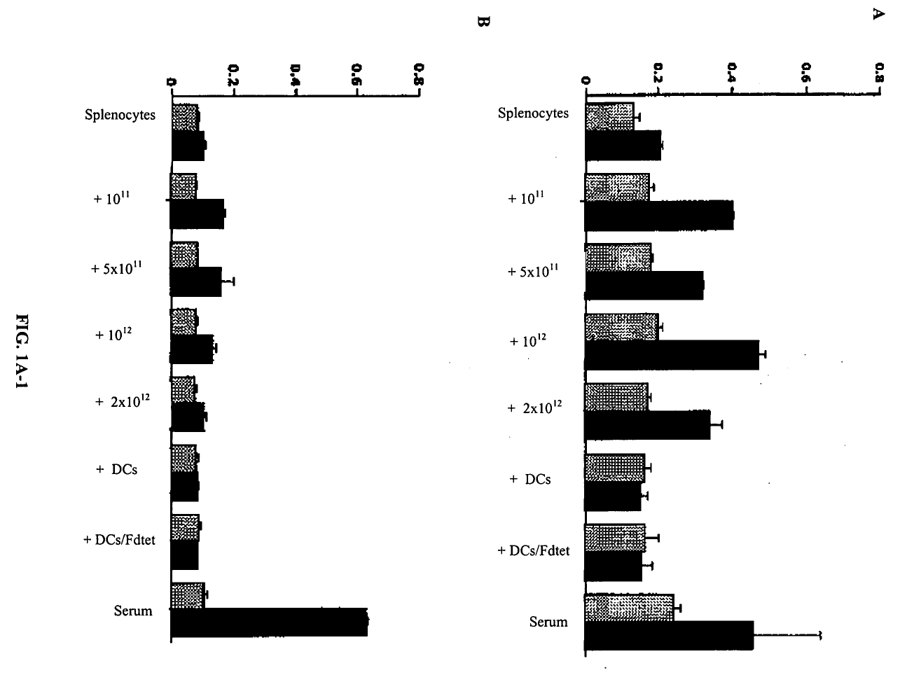

Results of in vitro immunization against Fd-Tet are shown in FIGS. 1A and 1B. ELISA plates were coated overnight at 4° C. with either pIII purified protein (5 μg/well) or Fd-Tet (10¹¹ TU/well). Conditioned media from the indicated wells, were collected 7 days after “first immunization” and 4 days after “second immunization.” As controls, pre and post immune serum from one animal inoculated with Fd-Tet (3 injections) were used. Plates were developed with anti-murine total Ig HRP conjugated (ZYMED) and OPD.

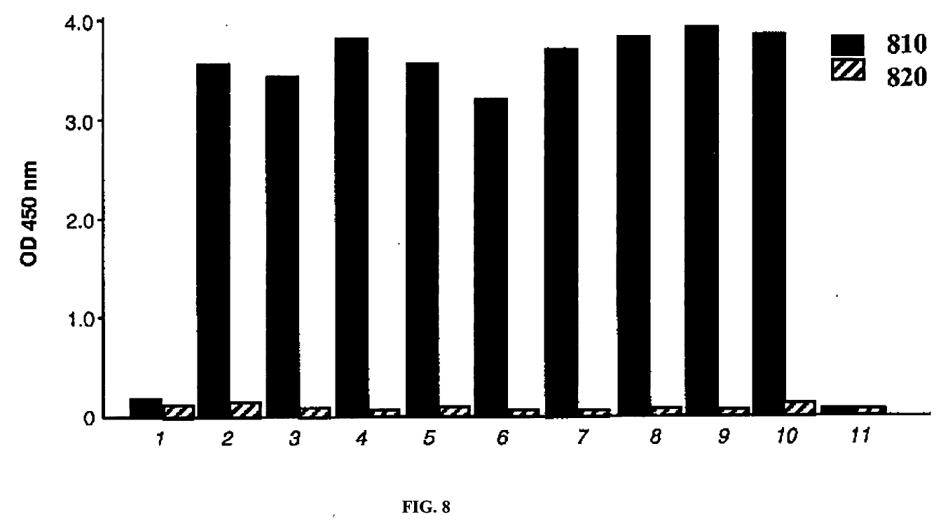

H-2K^{b}-tsA58 mice were immunized with a defined antigen (filamentous phage) and anti-phage antibody titers in the serum were monitored by ELISA. Anti-phage IgG titers reached high levels (OD₄₅₀>3 at 1:3,200 dilution, compared to <0.1 for pre-immune serum) seven days after a final boost (FIG. 8). Further testing of serial dilutions revealed that IgG titers against phage were on average about 1:6,400. Moreover, the serum titers against the pIII protein (coated at 10 μg/well) were on average about 1:1,600 (data not shown). Mouse spleens were collected and cell suspensions prepared in DMEM. The cells were distributed in 96-well plates and cultured at 33° C. The culture medium was changed completely three times during 2-3 weeks. Clones were observed in >90% of the wells after 3 weeks. To detect antibody reactivity, ELISA was performed with supernatants in microtiter well plates coated with phage particles. Up to 58% of the clones were positive for IgG reactivity against phage.

It was observed that splenocytes were healthy despite low cell density, and yielded robust levels of reactivity in supernatants from the majority of the wells. To obtain monoclonal lines, cells from positive wells were cloned by limiting dilution and most clones remained positive. Sub-cloning of monoclonal lines was repeated twice and virtually all of the resulting clones were positive, providing strong evidence that the lines generated were indeed derived from single clones.

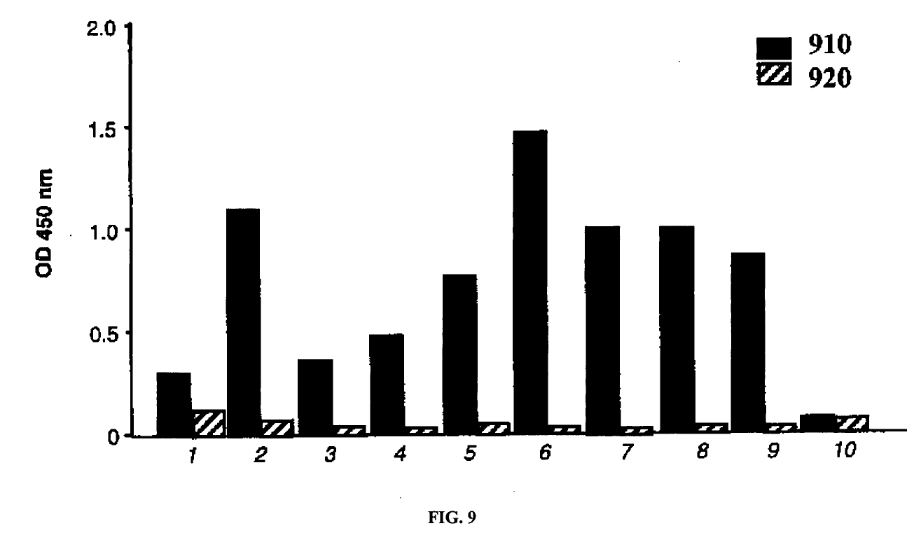

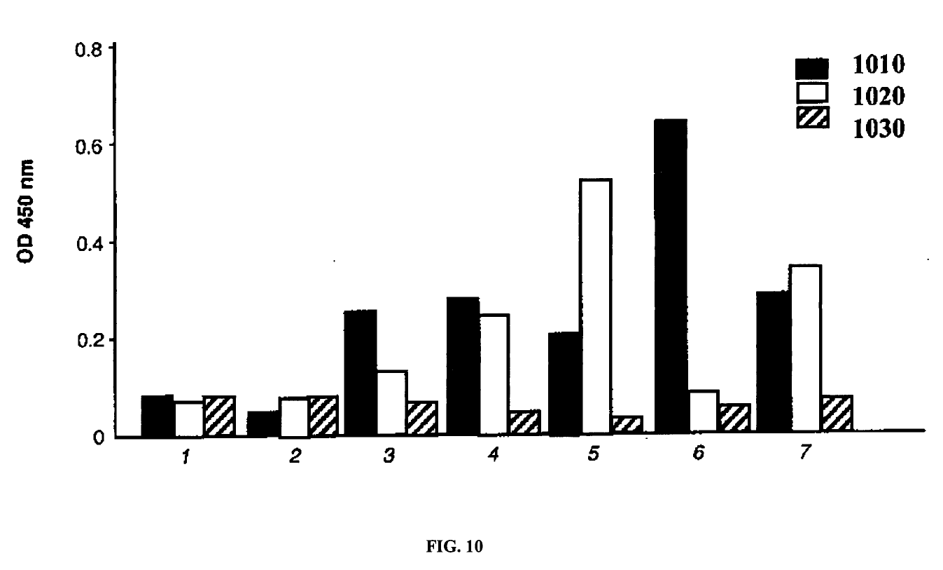

Clones emerging after 4-8 weeks were tested by ELISA against the phage particles and against the minor phage capsid protein (pIII). Again, most positive clones continued to react when expanded from 96-well to 24-well plates or after freeze and thaw (FIG. 9). Strong reactivity was observed against intact phage and some clones also reacted against recombinant pIII fusion protein (FIG. 10). Original plates and clones at all stages were kept in culture for up to 3 months.

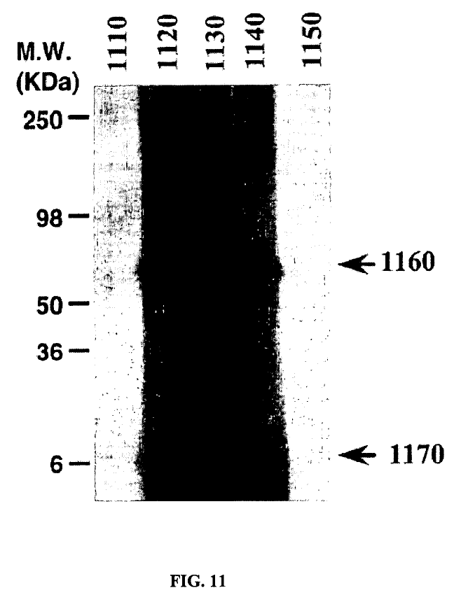

To determine whether antibodies screened by ELISA can recognize specific proteins in Western Blots, supernatants were evaluated from H-2K^{b}-tsA58 transgenic mouse-derived immortal splenocytes against the pIII and pVIII phage capsid proteins by resolving a filamentous phage preparation by SDS-PAGE. PVDF membranes containing phage proteins were incubated with pre-immune serum, post-immune serum, a commercially available anti-phage, or supernatants containing anti-phage IgGs secreted from immortal splenocyte clones. Cell culture media alone was used as an additional negative control. Antibodies reacting specifically with bands corresponding to the pIII and the pVIII phage capsid proteins were detected in the supernatants from H-2K^{b}-tsA58 transgenic mouse-derived immortal splenocytes (FIG. 11). This result demonstrates that antibodies produced by the methodology described here can also be used in applications such as immunoblotting (FIG. 11) or fluorescence activated cell sorting (FACS) of cell surface antigens (data not shown).

It appears that splenocytes from H-2K^{b}-tsA58 transgenic mice can yield high titers of IgG against defined antigens. This cell culture system ensures a reliable and reproducible source of monoclonal antibodies and eliminates the need for hybridoma generation.

Several advantages of the invention merit further comment. First, the antibody-synthesizing cells are stable for months and possibly years in culture, tolerate limiting dilution cloning, and freeze-thaw techniques without loss or inactivation of antibody production. Polyclonal populations have been frozen and viable clones are recovered that secret a given IgG (data not shown).

Second, immortal clones grow slowly at 33° C. and are genetically stable, allowing for timely processing of large number of samples (and, logically, the possibility of obtaining “rare” antibodies). H-2K^{b}-tsA58 -derived splenocytes enable the production of large amounts of specific polyclonal IgGs from wells containing clones that have been cultured long term. In contrast, hybridomas are problematic because in a random mixture of clones, non-secreting clones generally will overtake the secreting ones. Preliminary data suggest that the proliferation rate between IgG secreting and non-secreting splenocytes derived from an H-2K^{b}-tsA58 transgenic mouse is similar (unpublished observations).

Third, in vitro immunization is enhanced through the presence of other spleen-derived immortal cell types--such as macrophages and fibroblasts—that facilitate antibody production, whereas in vitro immunization is inefficient with mortal splenocytes or hybridomas. Given the recent restrictions placed on ascites production, this new technology favors convenient large-scale manufacture of monoclonal antibodies ex-vivo.

Fourth, crossing H-2K^{b}-tsA58 mice with mice expressing the genetic complement for human antibody production may also enable production of human monoclonal antibodies. The strategy described herein may replace hybridoma generation and streamline the production of mouse and human monoclonal antibodies, with profound and immediate scientific and medical benefits.

Results of ex vivo immunization using fd-Tet are shown in FIGS. 1A and 1B. ELISA plates were coated over night at 4° C. with either the pIII phage capsid protein (5 μg/well) or intact phage particles (10¹¹ TU/well). Conditioned media from cultured cells under different experimental conditions was collected at day 11 and 22 (spleen I) 19 and 26 (spleen II) after priming. As positive and negative controls for phage reactivity, pre- and post-immune anti-phage polyclonal serum was used. The serum derived from mice immunized with fd-Tet phage every other week for 12 weeks was collected. Plates were developed with a secondary anti-mouse Ig-peroxidase (ZYMED) and developed with TMB (Calbiochem). Optical density was monitored in an ELISA reader.

Morphology of Immortal Splenocytes

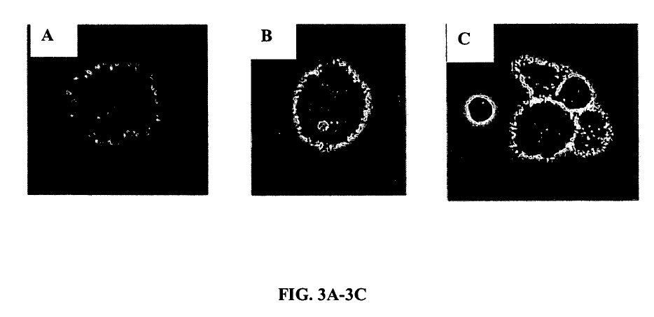

General morphology of immortal splenocytes from immunized animals are shown in FIGS. 3A, 3B and 3C. Pictures were taken after 2 months in culture. Follicular dendritic cells, clones of plasmocytes (producing antibodies B cells), macrophages and still unidentified epithelial-like cells (probably reticular epithelial cells) can be observed.

Morphology of Splenocytes from an Immunized Mouse

Spenocytes derived from an immunized mouse were analyzed visually after two months in culture. Several different cells were observed, for example follicular dendritic cells, clones of plasmocytes (producing antibodies B cells), macrophages and still unidentified epithelial-like cells (probably reticular epithelial cells).

Although spleen and bone marrow cell cultures have been demonstrated to work well for antigen presentation, data indicates that the lymph nodes also work well for antigen presentation (data not shown).

### Example 2

### Generation of Immortal Dendritic Cells (DCs) from Bone Marrow (BM)

In a parallel study, splenocytes were primed by co-incubation with dendritic cells (DC) previously exposed to phage (loaded with phage) or other antigens.

A. Methods

Harvesting and Isolation of Bone Marrow Cells

Bone marrow (BM) was harvested from the long bones of the femur, tibia and epiphysis of H-2K^{b}-tsA58 mice, by introducing a 27 G needle in the marrow cavity. Red blood cells were lysed with ammonium chloride. A single cell suspension was plated on petri dishes. Cells were incubated at 33° C. Each plate received 7 ml of RPMI 1640 with 10% FBS supplemented with murine (mu)GM-CSF (10 ng/ml) and r-muIL-4 (10 ng/ml). Three days later, plates were supplemented with 3 ml of complete media plus cytokines. After 5 days in culture, approximately 50% of the cell population was represented by immature dendritic cells. Loosely adherent proliferating DC aggregates were collected and replated. Immature DCs were co-incubated with either filamentous phage (fd-tet) (1.5×10¹² TU/1×10⁶ immature DCs) or apoptotic B16-F10 cells (2:1, DCs/B16). Incubation continued for 48 hr in the presence or absence of TNF-α (a factor known to induce DCs maturation).

Preparation of Apoptotic B16-F10 Cells

Apoptosis was induced in B16-F10 cells by applying UV irradiation (UV Stratalinker, Stratagene 4 joule/cm² for 20 min) to a suspension of approximately 1×10⁶ cells/ml of B16-F10 cells. After 24 hrs, 67% of the cells were Annexin positive that indicated the cells became apoptotic.

Induction of T Cell or B Cell Responses

In order to induce a T and/or B cell mediated response, DC cells may be mixed with spleen-derived cells. Next, DCs under different experimental conditions (loaded or not with antigens, e.g., phage or apoptotic cells), were co-incubated with isolated spleen-derived cells (SDC) in order to induce T and/or B cell-mediated responses in a ratio of 5:1, SDC/DCs.

B. Results

Dendritic Cells

The standard behavior of dendritic cells in vitro is altered in the conditionally immortal DC cells, based on their properties and response to biological factors. There may be selective pressure for B cells that respond to an antigen that is being constantly presented by dendritic cell in the same well. Ex vivo immunization may also be included in various embodiments of the invention to provide for constant antigen presentation.

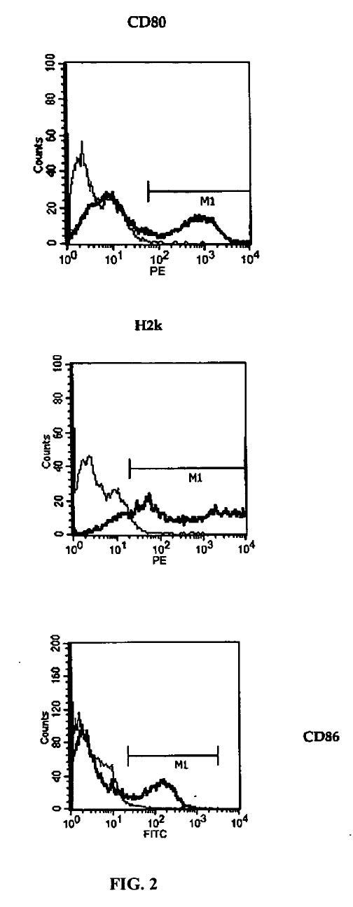

The presence of specific cell surface antigens in the “immortal” immature DCs may be evaluated by FACS analysis. Five days after plating, cells were evaluated for surface antigens using several antibodies, anti-CD80, anti-CD86, and anti-H2k (BD) antibodies (Weigel et al., 2002). (FIG. 2). The characteristic morphology of DCs differentiated with cytokines from bone marrow is shown in FIG. 2.

Stem Cells

Another use for immortalized cells may be in replacing the stem cell population of a diseased subject. In one example, one might irradiate the marrow of an animal such as a mouse, and replace it with the marrow or any source of stem cells from an immortalized animal such as an ImmortoMouse. To analyze any results, the normal animal may be sacrificed to identify any stem cells that may be immortal, which came from the immortomouse. This will allow the identification of specific organ homing stem cells. Also, crossing an ImmortoMouse with a Rosa mouse (expressing Lac Z in all cells) will also help in not only growing but tagging the cells coming from the ImmortoMouse to allow tracking in the recipient.

Brain stem cells from the ImmortoMouse have been isolated and characterized. The smallest cells growing by the round cells in FIG. 5, lowest right panel, appear to be spleen stem cells. Thus, isolation of immortalized stem cells and introduction of this population may be used in the future to improve the chances of survival of compromised subjects such as cancer patients.

### Example 3

### Generation of Monoclonal Antibodies Against Intact Cells

A. Methods

Immunization

H-2K^{b}-tsA58 mice (Charles River Laboratories, Wilmington, Mass.) were immunized with 5×10⁶ Mesenchymal Stem Cells (MSC) every other week for 3 weeks. ELISA and FACS were used to evaluate anti-MSC antibody titers in the serum. Animal experiments involved standard established procedures reviewed and approved by the Institutional Animal Care and Use Committee from the University of Texas M. D. Anderson Cancer Center.

Derivation of Immortal Splenocytes

Mice were sacrificed and their spleens were collected in Dulbecco's modified Eagle's medium (DMEM). Cells were released by gentle pressure applied to the capsule of the organ, which was placed between two frosty glass slides. Next, splenocytes were re-suspended in 15 ml of hybridoma medium with 10% CPSR plus hybridoma-enhancing supplements. Tissue debris were cleared by filtration through nylon mesh. The cells were re-suspended in 55 ml of culture medium and distributed in 6-, 24-, and 96-well plates at different densities. The plates were incubated at 33° C. The culture medium was changed completely three times during 2-3 weeks. Clones were observed in the majority of the wells.

The plating scheme was as follows: the spleen was re-suspended in 55 ml; one 6-well and one 24-well plate were seeded, the remaining cells were diluted in approximately 280 ml and distributed in 20×96 wells, 5×24 wells and 3×6 wells. The inventors evaluated by ELISA 59 clones from 96 wells plates (83% were +), and 61 clones from 24 (82% were +), 3 months into the study. It is clear that seeding from 55 ml in 24-well plates is the best possible culturing conditions in this system. Cells look healthy after months and all of the wells are positive. Sub-cloning from such wells can be achieved successfully.

### Example 4

### Anti-Tumor Reactivity Against Kaposi Sarcoma (KS) Cells and Mesenchymal Stem Cells (MSC)

A. Methods

Serum Collection

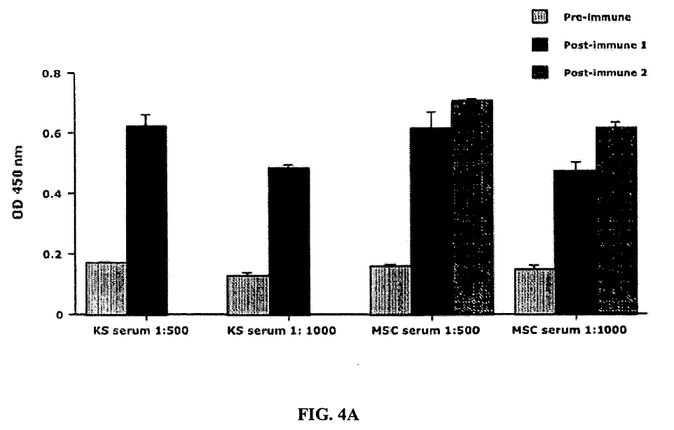

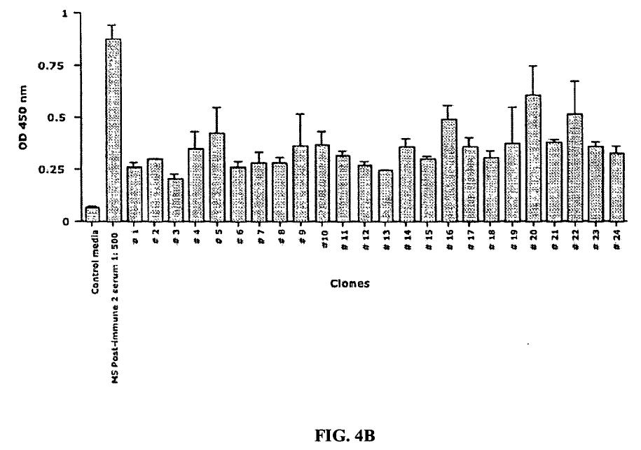

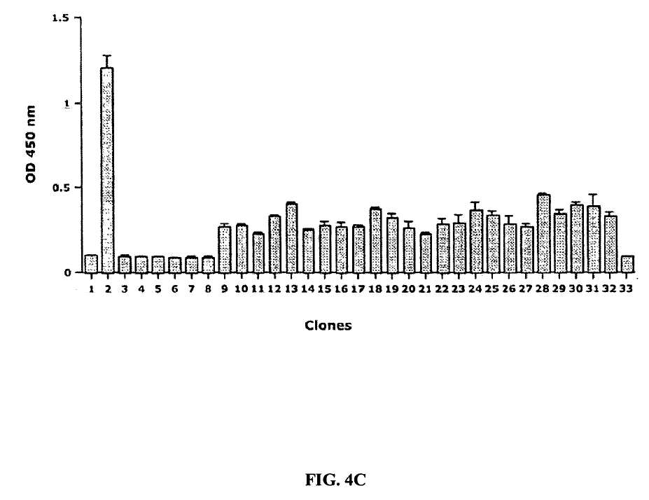

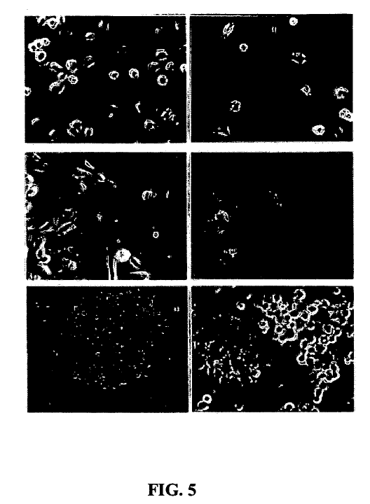

Mice were bleed before starting immunization protocol and after 2 or 3 weeks (Post immune serum 1: after 2 injections and Post immune serum 2: after 3 injections). Serum specific reactivity was assayed against KS and MSC cells plated in multiwell plates and fixed with PAF 2% as described bellow (FIG. 4A).

ELISA Assay

Serum anti-tumor reactivity was measured by ELISA. 3×10⁴ exponentially growing KS (FIGS. 4A and 4B) or 1.5×10⁴ MSC cells/well (FIG. 4C) were plated in a 96 well plate. After overnight incubation at 37° C., cells were washed once with PBS, fixed in 2% PFA for 10 min at room temperature and rinsed once with PBS. Plates were preserved at −20° C. until use. After blocking with PBS-2% BSA for 1 h at RT, serum dilutions 1/500 or 1/1000 in PBS-0.5% BSA (FIG. 4A), were added in duplicates and incubated overnight at 4° C. Antibodies were plated directly from culture supernatants (FIGS. 4B and 4C). The following day, the plates were rinsed three times with PBS-0.5% BSA, 0.01% Tween 20 and once with PBS only. 100 μl of a 1/2000 dilution of rabbit-antimouse Ig horseradish peroxidase (HRP) conjugated (ZYMED Laboratories Inc., California, USA) in PBS-0.5% BSA were added to the plates. After 90 min incubation at room temperature with shaking, the plates were washed three times as above. The reaction was developed with ortho-phenylenediamine (OPD) (Sigma-FAST, Sigma-Aldrich, St. Louis, USA Fast-tabs) and stopped with 50 μl/well of 3 M sulfuric acid. Absorbance was read at 450 nm in a Microplate Reader. More than 100 clones were evaluated.

Subcloning of Positive Cells

Cells from the positive wells were sub-cloned by limiting dilution (0.1 or 0.5 cells per well in 96-well plates) in order to obtain monoclonal lines.

### Example 5

### Generation of Immortal Thymocytes

The thymus was removed from a H-2K^{b}-tsA58 at day 14 and collected in Dulbecco's modified Eagle's medium (DMEM). Cells were released by gentle pressure applied to the capsule of the organ, which was placed between two frosty glass slides. Next, thymocytes were re-suspended in 15 ml of hybridoma medium with 10% CPSR plus hybridoma-enhancing supplements. Tissue debris was cleared by filtration through 70 μm nylon mesh. The cells were distributed in 24-well plates and cultured at 33° C.

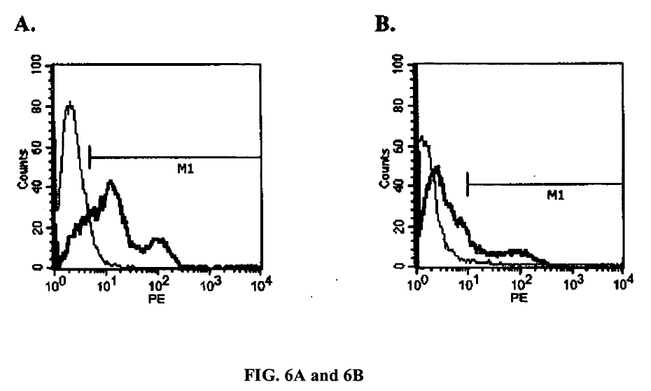

Remaining cells were analyzed by FACS, using anti murine antibodies: CD3, B220, H2k, CD86, CD80, CD11c and MAdCAM-1 (that recognizes epithelial reticular cells). The FACS results showed the following percentages of positive cells (FIGS. 6A and 6B) CD3+: 75% and H2k+: 20%, respectively. CD45RA (B220): 3.5% (data not shown) CD86+: 4% (data not shown) CD80+: 0% (data not shown) CD11c+: 0% (data not shown) MAdCAM-1+: 0% (data not shown).

All of the methods, compositions and apparatus disclosed and claimed herein can be made and used without undue experimentation in light of the present disclosure. It will be apparent to those of skill in the art that variations may be applied to the methods, compositions and apparatus described herein without departing from the concept, spirit and scope of the claimed subject matter. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the claimed subject matter.

### References

The following references, to the extent that they provide exemplary procedural or other details supplementary to those set forth herein, are specifically incorporated herein by reference.

U.S. Pat. No. 5,270,191

U.S. Pat. No. 5,685,570

U.S. Pat. No. 5,688,692

U.S. Pat. No. 5,866,759

U.S. Pat. No. 5,939,598

U.S. Pat. No. 6,075,181

U.S. Pat. No. 6,114,598

U.S. Pat. No. 6,150,584

U.S. Pat. No. 6,162,963

U.S. Pat. No. 6,165,745

U.S. Pat. No. 6,235,883

U.S. Pat. No. 6,399,384

U.S. Pat. No. 6,602,503

U.S. Pat. No. 6,657,103

U.S. Pat. No. 6,673,986

Barbas et al., In: Phage Display: A Laboratory Manual (Cold Spring Harbor Lab. Press, Plainview, N.Y.), 2000.

Breitling and Dubel, Recombinant Antibodies, Wiley Publishers, 1999.

Daniell et al., Trends Plant Sci., 6:219-26, 2001.

Dykman et al., Journal of Microbiological Methods, 247-248, 1996.

Harlow and Lane, In: Antibodies: A Laboratory Manual, Cold Spring Harbor Laboratory, Cold Spring Harbor, N.Y., 346-348, 1988.

Harlow and Lane, In: Antibodies: A Laboratory Manual, Cold Spring Harbor Laboratory, Cold Spring Harbor, N.Y., 346-348, 1988.

Jat et al., Proc. Natl. Acad. Sci. USA, 88:5096-5100, 1991.

Johnson et al., In: Biotechnology And Pharmacy, Pezzuto et al. (Eds.), Chapman and Hall, NY, 1993.

Karpas et al., Proc. Natl. Acad. Sci. USA, 98:1799-1804, 2001.

Kohler and Milstein, Nature, 256:495-497, 1975.

Mintz et al., Nat. Biotechnol,.21(1):57-63, 2003.

Weigel et al., Blood, 100:4169-4176, 2002.

Winter and Milstein, Nature, 349:293-299, 1991.

Winter et al., Annu. Rev. Immunol., 12, 433-455, 1994.

Zacher et al., Gene, 9:127-132, 1980.

What is claimed:

1. A method for generating an antibody-producing cell that produces an antibody to a desired antigen comprising the steps of:
(a) contacting antibody-producing cells with the desired antigen in a manner effective to induce the cells to produce antibodies against the antigen, wherein the antibody-producing cells are capable of being immortalized without forming hybridomas; and
(b) immortalizing the antibody-producing cell.

2. The method of claim 1, wherein the antibody-producing cell comprises a transforming oncogene that is conditionally functional or conditionally expressed, and the immortalization is effected by inducing said transforming oncogene.

3. The method of claim 2, wherein the conditionally functional transforming oncogene is a temperature sensitive SV40 Large Tumor antigen (tsSV40Tag).

4. The method of claim 3, wherein the tsSV40Tag is an A58S-SV40Tag.

5. The method of claim 4, wherein the transforming oncogene is induced by culturing the antibody producing cells at from 25° C. to 35° C.

6. The method of claim 5, wherein the antibody producing cells are cultured at from 30° C. to 35° C.

7. The method of claim 6, wherein the antibody producing cells are cultured at 33° C.

8. The method of claim 5, wherein said antibody-producing cells are cultured in hybridoma culture medium.

9. The method of claim 5, further comprising assessing the antibody producing capabilities of said antibody-producing cells.

10. The method of claim 9, wherein said assessing comprises assaying antibody binding to said desired antigen.

11. The method of claim 1, wherein single cells are selected and cultured to produce a monoclonal population that produce monoclonal antibodies.

12. The method of claim 11, wherein single cells are selected by dilution cloning.

13. The method of claim 1, wherein multiple cells are selected and cultured to produce a polyclonal population that produce polyclonal antibodies.

14. The method of claim 1, wherein the antibody-producing cell comprise spleen cells.

15. The method of claim 1, wherein the antigen is selected from the group consisting of peptides, proteins, glycoproteins, lipoproteins, carbohydrates, viruses, bacteria, pathogenic microorganisms, tissue, whole cells, biopsy tissue, patient-derived cells, tissue extracts, fresh or cultured tissues, apoptotic cells, subcellular components, membrane, cytoplasm, and nuclear fractions from cells and tissues, purified proteins, partially purified proteins, laser captured tissue, paraffin embedded and fixed tissue.

16. The method of claim 15, wherein the tissue comprises subject-derived tumor tissue.

17. The method of claim 1, wherein the antibody-producing cells are obtained from a transgenic mouse having antibody-producing cells that are capable of being immortalized without forming hybridomas.

18. The method of claim 17, wherein the transgenic mouse comprises the genetic complement for producing human antibodies.

19. The method of claim 1, wherein the antibody-producing cells are comprised in a mouse, and the selected antigen is administered to the mouse in a manner effective to induce the antibody-producing cells to produce antibodies.

20. The method of claim 1, wherein contacting with the desired antigen comprises co-culturing the antibody-producing cell with an antigen presenting cell.

21. The method of claim 20, wherein the antigen presenting cell is a dendritic cell.

22. The method of claim 1, wherein in the antibody-producing cell comprises the genetic complement for human antibody production.

23. The method of claim 1, further comprising purifying antibodies produced by said antibody-producing cells.

24. The method of claim 23, further comprising administering said antibodies to a subject in need of therapeutic antibodies.

25. A method for generating an antibody-producing cell that produces a human antibody to a desired antigen comprising the steps of:
(a) obtaining an antibody-producing cell that conditionally expresses a transforming oncogene or expresses a conditionally functional transforming oncogene and expresses the genetic complement for human antibody production, wherein immortalization of said antibody-producing cell is effected by inducing the expression or function of said transforming oncogene; and
c) contacting said antibody-producing cells with the desired antigen in a manner effective to induce the cells to produce human antibodies against the antigen, wherein said antibody-producing cells are capable of being immortalized without forming hybridomas; and
(b) immortalizing said antibody-producing cell.

26. The method of claim 25, wherein the conditionally functional transforming oncogene is a temperature sensitive SV40 Large Tumor antigen (tsSV40Tag).

27. The method of claim 26, wherein the tsSV40Tag is an A58S-SV40Tag.

28. The method of claim 27, wherein the transforming oncogene is induced by culturing the antibody producing cells at from 25° C. to 35° C.

29. The method of claim 28, wherein the antibody producing cells are cultured at from 30° C. to 35° C.

30. The method of claim 29, wherein the antibody producing cells are cultured at 33° C.

31. The method of claim 25, wherein single cells are selected and cultured to produce a monoclonal population that produce monoclonal antibodies.

32. The method of claim 25, wherein multiple cells are selected and cultured to produce a polyclonal population that produce polyclonal antibodies.

33. The method of claim 25, wherein the antibody-producing cell comprise spleen cells.

34. The method of claim 25, wherein the antigen is selected from the group consisting of peptides, proteins, glycoproteins, lipoproteins, carbohydrates, viruses, bacteria, pathogenic microorganisms, tissue, whole cells, biopsy tissue, patient-derived cells, tissue extracts, fresh or cultured tissues, apoptotic cells, subcellular components, membrane, cytoplasm, and nuclear fractions from cells and tissues, purified proteins, partially purified proteins, laser captured tissue, paraffin embedded and fixed tissue.

35. The method of claim 34, wherein the tissue comprises subject-derived tumor tissue.

36. The method of claim 25, wherein the antibody-producing cells are obtained from a transgenic mouse having antibody-producing cells that are capable of being immortalized without forming hybridomas.

37. The method of claim 25, wherein the antibody-producing cells are comprised in a mouse, and the selected antigen is administered to the mouse in a manner effective to induce the antibody-producing cells to produce antibodies.

38. The method of claim 25, further comprising purifying antibodies produced by said antibody-producing cell.

39. The method of claim 38, further comprising administering said antibodies to a subject in need of therapeutic antibodies.
